(19) United States
(12) Patent Application Publication
(10) Pub. No.: US 2024/0049692 A1
(43) Pub. Date: Feb. 15, 2024

(54) MIXED SOLID WASTE PROCESS

(71) Applicant: ARC Ento Tech Ltd, Mardi (AU)

(72) Inventors: Ramon Manuel Centenera Atayde, Mardi (AU); Ricardo Martin Atayde, Hornsby Heights (AU)

(21) Appl. No.: 18/246,829

(22) PCT Filed: Feb. 17, 2021
(86) PCT No.: PCT/AU2021/050134

§ 371 Date: Mar. 27, 2023

(30) Foreign Application Priority Data
Oct. 14, 2020 (AU) 2020903724

(51) Int. Cl. A01K 67/033; B09B 3/35; B09B 3/40

(52) U.S. Cl. CPC A01K 67/033; B09B 3/35; B09B 3/40; A01K 2227/706

(57) ABSTRACT

The invention provides an integrated solution to the processing of the major portion of household waste comprising organic matter and plastics in an integrated way that produces useful products from both streams.

### PRIORITY TO RELATED APPLICATIONS

This application is a national phase entry of, and claims the benefit and priority to, International Patent Application No. PCT/AU2021/050134, filed Feb. 17, 2021, and entitled “MIXED SOLID WASTE PROCESS,” which claims the benefit of, and priority to Australian Application No. 2020903724, filed Oct. 14, 2020, entitled “MIXED SOLID WASTE PROCESS,” the contents of which are incorporated by reference in their entirety.

### TECHNICAL FIELD

The invention relates to the field of municipal waste processing. In particular, the invention relates to the integration of a method of processing mixed solid waste comprising organic matter using dipteran larvae, and high protein biomass (in the form of advanced dipteran larvae) obtained therefrom; and a process of processing plastic material from said waste into a metallurgical reductant material.

### BACKGROUND OF THE INVENTION

One of the most pressing global issues of today is the generation and disposal of mixed solid waste and its impact on the environment. The problem emanates from the fact that consumption exceeded what nature can breakdown safely on its own. This has caused a backlog of unprocessed waste stockpiles in landfills causing a number of serious environmental issues.

The issue with mixed solid waste is that its bulk requires land space to store to allow it to reduce by natural processes. Unfortunately, the time to reduce the bulk and free the space is far exceeded by the amount of mixed solid waste generated. The result is the need to develop and operate more areas for landfill purposes to handle and store the waste. As a result, there is an economic and environmental cost to this continuing requirement for new landfill areas in a world where land values and urban development is limiting available sites for landfill operations.

The composition of typical mixed solid waste is of the order of about 50% biodegradable organics, about 30% recyclable, about 20% residual and a low, but often present is an amount of special/hazardous content.

The decomposition of organic material in mixed solid waste generated by households decomposes and produces methane, a major greenhouse gas attributed to other global environmental problems. The majority of landfill sites around the world do not have effective process to manage the problem and rely mostly on natural decomposition of organic matter after burying at the site. This continues to put a strain on land, water and air assets of the community.

Organic materials come in the form of digestible organics such as waste food and indigestible organics such as wood and organic rubber materials. This forms the bulk of mixed solid waste that if not processed are buried in landfills. All organics eventually decompose into compost material. However, the natural process when buried also generates a number of pollutants such as methane and acid leachates.

Many concepts and solutions have been tried but none have been able to deliver a total processing solution. The main issue lies in two major components of mixed solid waste that do not go through the recycling process, plastics and organics.

Thus, there is a need for improved manners of processing mixed solids waste to reduce their volume, and to minimise and/or reduce their impact on the environment, especially processes which result in a value-added product, such as a high-protein alternative stock feed.

Further, since the 1950s around 8.3 billion tonnes of plastic have been produced and consumed worldwide (2014 data). Over the past 50 years plastic production has grown exponentially to cope with increased demand by day to day applications for everyday living. This demand and consumption is likely to continue to rise alongside urban growth and global consumption trends.

Out of the seven types of plastics representing all plastic materials produced today only three can be recycled, with the balance going to landfill for burying or to incinerators for destruction.

It is estimated that over a million plastic bottles are bought and disposed of around the world every minute. Approximately only 13% of used water bottles are recycled each year. In addition, it is estimated that over 2 million plastic bags are used every minute and thrown away.

Plastic waste requires a significant amount of time to break down naturally. However, as it decomposes it forms micro plastics which contaminate both land and water.

On average, the typical household mixed solid waste contains over 52% organic material. The decomposition of organic material in mixed solid waste generated by household produces methane, a major greenhouse gas associated with other global environment problems.

The majority of landfill sites around the world do not have effective processes to manage the problem and rely mostly on natural decomposition of organic matter after burying at the site. This continues to put strain on land, water and air assets of the community.

The most common accepted principle applied in mixed solid waste management today are the 3Rs (Re-use, Reduce, Recycle). It has been effective in part but has failed to provide a comprehensive solution that resolves the entire problem.

Accordingly, it is an object of the invention to provide processes that ameliorate at least some of the problems associated with the existing solid waste management issues re disposal of organic and plastic waste material.

### SUMMARY OF THE INVENTION

The invention, in an overarching sense, provides an integrated solution to the processing of the major portion of household waste comprising organic matter and plastics. The invention provides a method of processing these materials in an integrated way that produces useful products from both streams.

The present investigations have surprisingly shown that mixed solids waste (MSW) can be at least partially processed effectively and efficiently using dipteran larvae, significantly reducing the organics load of the MSW, converting it to high-protein insect larval biomass which is readily consumed as it is by stock such as fish and poultry, and which can also be converted to fertiliser, pelleted feed and other forms of proteinaceous feed materials.

Thus, according to an aspect of the invention, there is provided a continuous process for treatment of mixed solids waste to reduce its organics load, wherein the waste comprises from at least 10% to up to 80% biodegradable organic matter, said method comprising introducing mixed solids waste and dipteran larvae into the entrance of a digester and collecting treated reduced organics waste at the exit of the digester, optionally harvesting dipteran larvae at one or more desired locations along the digester.

The inventors have devised a method for the continuous processing of the organic matter in the mixed solids waste, compared with the batch processing of such waste in the prior art. This allows the process to be integrated with a method for processing plastic materials in the waste into other useful products.

In certain embodiments the dipteran larvae are of a species of the Stratiomyidae family of flies, and in particular embodiments the dipteran larvae are of black soldier flies (Hermetia illucens).

According to a particular embodiment of the present invention there is provided a continuous process for treatment of mixed solids waste to reduce its organics load, wherein the waste comprises from at least 30% to up to 70% biodegradable organic matter and from about 20% to about 40% digestible organic matter, said method comprising introducing mixed solids waste into the entrance of a substantially cylindrical digester at a rate of between 0.1 and 0.5 volumes per day, wherein said digester is disposed at a descending angle of between 1° and 5° and is axially rotated at a speed of between 2 and 40 revolutions per day, and wherein the digester is inoculated at the entrance of the digester with black soldier fly larvae which are at least 1 mm long, at least 3 days old, or both at least 1 mm long and at least 3 days old, wherein said larvae are inoculated at a rate of between 10 and 25EGRL/T digestible organic matter, and collecting treated reduced organics waste at the exit of the digester, and harvesting black soldier fly larvae at the entrance of the digester and from the treated waste exiting the exit end of the digester.

In certain embodiments of processes according to the present invention, the dipteran larvae are continuously harvested from the digester, and in certain embodiments the larval biomass may be harvested from the entrance of the digester as well as from the treated waste at the exit of the digester.

Dipteran larval biomass, such as black soldier larval biomass, obtained from a process of the invention is also provided. Also provided is organic matter treated by a process of the invention (i.e. residue left over after digestion by the dipteran larvae, and effectively in the form of composted material), which may be used for horticultural applications.

As used herein the term “MSW” means mixed solids waste.

As used herein the term “BSF” means black soldier fly.

According to another aspect of the invention, there is provided a method of producing a metallurgical reductant material from unsorted municipal waste that has optionally passed through the process of organic digestion described above, and including at least 10% plastic material or the like, said method incorporating the steps of: shredding said waste material; subjecting said shredded waste to a process of partial pyrolysis at temperature of between 100° C. and 200° C. in the substantial absence of oxygen; subjecting said partially pyrolysed waste to a process of extrusion at elevated pressure and temperature and in the substantial absence of oxygen; and size reduction of the extrudate into briquettes, pellets or the like.

Preferably, the method further incorporates the step of drying and semi-pyrolysing said waste material prior to the partial pyrolysis stage to remove excess moisture. Preferably, the process temperature is approximately 200° C.

Preferably, the extrusion temperature is between 100° C. and 280° C., depending on the gasification temperature of the polymer materials, and is preferably 200° C.

According to another aspect of the invention, there is provided a metallurgical reductant material produced via the process described above.

Now will be described, by way of a specific, non-limiting example, a preferred embodiment of the invention with reference to the figures.

### BRIEF DESCRIPTION OF THE FIGURES

FIG. 1 shows a digester layout according to the invention, including MSW feed, BSF inoculation, and separation of treated waste streams, including BSF larvae harvesting.

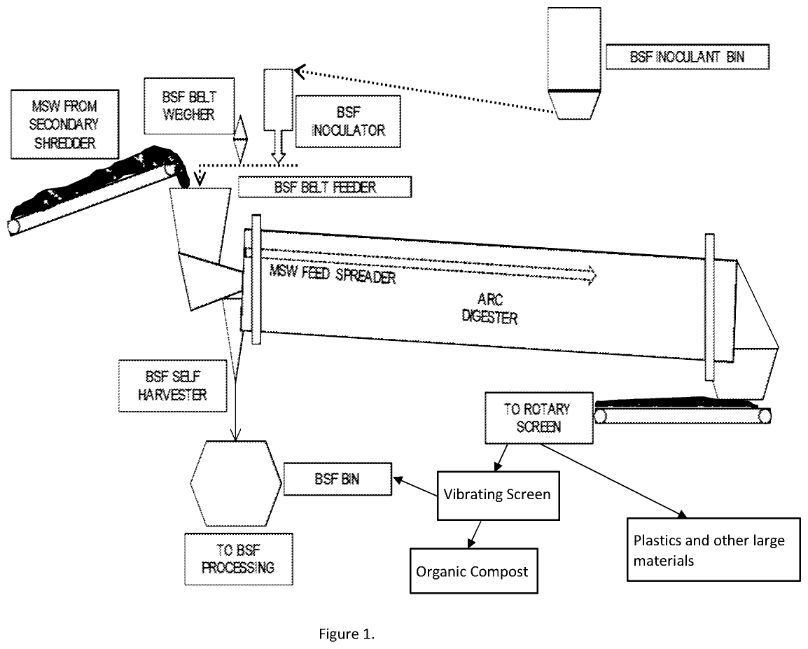

FIG. 2 provides a MSW treatment flowsheet according to a preferred process of the present invention.

FIG. 3 provides a more detailed factory MSW flowsheet treatment flowsheet according to a preferred process of the present invention.

FIG. 4 shows an RDR processing layout according to the invention.

FIG. 5 shows an RDR processing plant flowsheet.

FIG. 6 shows a landfill remediation flowchart incorporating processes according to the invention.

FIG. 7 shows a landfill remediation layout incorporating processes according to the invention.

FIG. 8 is a schematic of the mixed solid waste organic processing operation according to the invention.

FIG. 9 is a schematic of a landfill remediation process incorporating mixed solid waste processing and RDR processing that are integrated according to the invention.

### DETAILED DESCRIPTION OF THE INVENTION

As used herein, the terms “biodegradable organic matter” and “biodegradable organics” in the context of waste materials means materials of plant or animal origin such as vegetable or meat materials and including processed organic materials such as paper and cardboard.

As used herein, the term “treating” in the context of mixed solids waste and the biodegradable organic matter therein means subjecting the biodegradable organic matter to at least partial digestion by dipteran larvae, but not necessarily complete digestion.

The present invention will be described with reference to a particular implementation. It should be understood that the implementation discussed is purely illustrative and is no way limitative of the scope of the inventions disclosed. Various inventive features are disclosed, and it will be understood that this disclosure includes them in the combination as discussed, as well as their individual integers and in sub combinations.

Disposal of MSW generated by households, office spaces, markets, institutions, street litter, etc., is an ever-increasing problem, and typically requires ever increasing land spaces for dumping of the waste. This is not sustainable, and often also results in undesirable or even toxic leachates and gases being released from such sites.

A large portion of MSW comprises biodegradable organic matter—typical MSW may comprise, for example, 52% biodegradable organics, 28% recyclable materials (metals, glass, plastics), 18% residuals and about 2% special/hazardous materials. The composition varies between countries but in principle they have very similar profiles regardless of geographical location.

The most commonly accepted approach applied in mixed solid waste management today involves the 3Rs (Re-use, Reduce, Recycle). While this has been effective in part, it has failed to provide a comprehensive solution that resolves the entire problem.

The present invention aims to reduce this burgeoning problem by re-forming and re-purposing at least the biodegradable organic portion of MSW.

Composting of biodegradable organic waste of vegetable and (to a lesser extent) animal origin, such as agricultural and horticultural waste is well established, and is typically done by way of batch processes. Those methods typically employ primary degraders such as insect larvae, worms, or a combination of such degraders. Use of black soldier fly (Hermetia illucens) has been investigated significantly in the last 2 to 3 decades.

However, use of such composting methods for treatment of MSW has not been described, and commercial methods for dealing with MSW generally still rely on dumping/landfill, incineration, or a combination thereof. Importantly, commercial systems for treating large volumes of waste materials, such as MSW, need to be efficient and, ideally, continuous, which presents a problem for primary degraders in particular.

Through the present investigations it has been surprisingly found that by controlling the feed rates, aeration and mixing of MSW undergoing treatment, dipteran larvae, and especially BSF larvae, can thrive in a continuous process. Not only can they thrive, but grow and result in a value-added product themselves as a high protein feed material, particularly useful as stock feed.

### Mixed Organic Waste

Any mixed solids waste comprising sufficient biodegradable organic matter to sustain dipteran larvae may be used in processes according to the present invention. Typically this will require the MSW to comprise at least 10% organic matter. This is typically not an issue, as most MSWs, such as municipal solid waste streams, comprise about 50% biodegradable organic matter and between about 20% and 40% digestible organic matter. Although municipalities in certain countries, with significant recycling activity, may have slightly lower biodegradable organic matter and higher levels of soft plastic materials, or even vice versa, those values may be evened out over larger areas and, in principle, MSW streams will typically comprise close to 50% biodegradable organic matter. If a particular stream of MSW is particularly poor in biodegradable organic matter, it may be blended with other stockpiled MSW of a higher biodegradable organic matter content. Alternatively, factory policy of mixing all incoming streams of MSW should provide an effective means of evening out biodegradable organic matter in MSW fed into a digester vessel in processes according to the present invention.

Inclusion of non-biodegradable material in the digester has actually been found to be beneficial to the process, as this allows for breaking up of the material during digestion, allowing for better aeration, as well as better drying of the material.

Thus, MSW for feeding into continuous processes according to the present invention may comprise from at least 10% to up to about 80% biodegradable organic matter, such as from at least 20% to about 70%, at least 30% to about 70%, about 40% to about 60%, about 40%, about 45%, about 50%, about 55%, or about 60% biodegradable organic matter.

The MSW for feeding into continuous processes according to the present invention may comprise from at least 10% to up to about 60% digestible organic matter, such as from at least 15% to about 50%, at least 20% to about 40%, about 20% to about 35%, about 15%, about 20%, about 25%, about 30%, about 35%, or about 40% digestible organic matter.

The digestible organic matter content of MSW being fed into a digester vessel in a process according to the present invention may at least to some extent dictate the rate of inoculation of the digester with dipteran larvae, the mean residence time of the MSW within the digester, axial rotation speed of the digester, or any combination thereof, if the biodegradable organic matter in the MSW is to be fully treated, as will be further discussed below.

MSW may be treated prior to digestion by dipteran larvae to remove large indigestible and or recyclable materials, such as metals and glasses as well as, for example, large plastic, leather and wooden materials.

The MSW may also be processed through one, two or more shredders to reduce the size of particles in the MSW.

However, for the purpose of inventions according to the present invention processing can be minimal, although removal of metals and glasses is desirable.

Shredding, or milling to reduce particle size by other means, while desirable to reduce blockages in digesters and increase the surface of organics available to dipteran larvae, should also not be extensive, as this may reduce aeration throughout the MSW (larger particles will result in larger and more frequent air spaces), and excessive shredding may also result in plastic particles becoming inseparable from dipteran larvae and compost arising from digestion of organics by the dipteran larvae. Separation of dipteran larvae and digested organic waste is conveniently done by screening, where a screen mesh of between 25 mm and 10 mm will suitably separate non-digestible material (retained fraction) from dipteran larvae and digested organic material (screened/sieved material), where the MSW has been treated to have an average particle size (diameter and/or length/width) no smaller than 25 mm and no greater than 150 mm particle size, preferably no smaller than about 35 mm to about 50 mm.

Thus, according to certain embodiments of the present invention, the MSW for treatment by a process according to the present invention has had recyclable metal and glass materials substantially removed, optionally other large indigestible materials removed, and has been processed to have an average particle size (diameter and/or length/width) of no less than 25 mm, and no greater than 150 mm particle size, such as from about 35 mm to about 100 mm, from about 40 to about 80 mm, from about 50 mm to about 75 mm, about 30 mm, about 40 mm, about 50 mm, about 60 mm, about 70 mm, about 80 mm or about 90 mm. Such a particle size may be achieved by, for example, shredding through one, two or more shredder cycles where the smallest shredder jaw opening used is, for example, at least 50 mm, more preferably 75 mm, and even more preferably 100 mm, and the largest shredder jaw opening is about 200 mm.

### Dipteran Larvae

The order Diptera encompasses the true flies, the larvae of which are small, typically white, and may consume dead or alive vegetable or animal material. For the purposes of the present invention, which relates to treatment of large volumes of waste at a treatment facility, presence of fly larvae that may be undesirable in a domestic or agricultural situation (as they may, for example, be agricultural pests, or even attack live animals), is not necessarily a problem, although it is preferable that the larvae be of a fly species that is not a pest, and which predominantly consumes dead vegetable and animal matter.

Housefly (Musca domestica) larvae have been known to be used in composting, but perhaps the most studied fly larva that consumes vegetable matter is that of the black soldier fly (Hermetia illucens). This having been said, there is a very wide range of other members of the species Hermetia, as well as other soldier flies (of the family Stratomyidae, such as members of the genus Ptecticus, of which at least Ptecticus tenebrifer and Ptecticus melanurus are known to be effective in consumption of biodegradable organic matter), or other members of the order Diptera, and it is expected that suitable dipteran larvae for use in processes according to the present invention other than housefly larvae and BSF larvae will be readily identified and isolated by those skilled in the art. Combinations of dipteran larvae, which may be advantageous in treating different components of MSW, are also contemplated by the present invention.

According to an embodiment of processes of the invention, the digester is inoculated with larvae of the black soldier fly (BSF).

BSF larvae are very efficient and voracious waste converters with a high reproduction and growth rate. The fly lives most of its life as a larva, the larval stage lasting between ten and fourteen days, with the adult stage being only short, and during which the adult does not feed, but only reproduces and dies shortly afterwards (if it is unable to breed it can survive up to seven days before it dies of starvation). The fact that the adult fly does not feed is particularly useful as it prevents the adult fly from ingesting pathogens and spreading them as they feed on different organic waste material.

The Black Soldier Fly larvae can be used to produce an insect-based protein meal which is a viable and sustainable alternative to fishmeal used in most animal feeds. The majority of commercial fishmeal is sourced from wild fish stocks in our oceans. It is currently the primary source of protein in animal feed such as for poultry, hogs and aquaculture. However, as the global population grows the demand for food commensurately and puts pressure on natural food sources. This renders the supply of fishmeal from ocean stocks unsustainable. However, the production of insect meal from Black Soldier Flies larvae by processes according to the present invention relies on waste streams generated by human consumption. This creates a compatible cycle for both consumption and production where the more humans consume, the more the Black Soldier flies convert back into animal feed to produce food for consumption.

The Black Soldier Fly also has the added advantage of being resilient and adaptable in complex waste environments, unlike worms, which tend to be sensitive, and is readily found in most regions around the world (so it, or perhaps a close relative, can be locally sourced).

Although BSF larvae are tough and resilient, conditions in a continuous digester system, especially shear stresses resulting from axial rotation of the digester, place the larvae under stress. The larvae are sufficiently hardy for life in the digester when they first become properly visible, at about 1 mm in length, which occurs at about 4-5 days after hatching under ideal conditions, although this may take longer (up to 11 to 15 days) under conditions of lower temperature, lower feed levels, or other stressful conditions. When hatched under ideal conditions, larvae at least 4 days post-hatching, and preferably at least 5 days old are more resilient to the stresses in the digester and have been found to not only be able to survive the continuous digester system, but thrive in it, whereas younger larvae may struggle at least initially. Thus, for BSF larvae, ideally, the larvae are at least 0.5 mm long, such as 0.7 mm long, 0.8 mm long, 1 mm long, 1.1 mm long, 1.2 mm long, 1.3 mm long, 1.4 mm long, or 1.5 mm long when they are inoculated into the digester, which may mean they are about 3, about 4, about 5, about 6, about 7, about 9, or about 11 days post hatching. Optimal age of other dipteran larvae for inoculation into digesters may be expected to vary, but can be readily determined.

BSF larvae have the additional benefit that, when they are ready to pupate, they cease feeding and migrate up to and away from the surface of the substrate they are in. This occurs in the digesters, and a route is provided at the top of the digester for the maturing BSF larvae to migrate out of the digester, effectively self-harvesting. Some larvae also travel through the digester and come out the exit.

Dipteran larvae may be hatched in purpose-built hatchery facilities, preferably close by to the digester system, to ensure reliable and regular supply. Dipteran eggs may be obtained by allowing a certain amount of larvae to pupate, mature into adult flies and mate in the vicinity of suitable substrate and feed source. For BSF larvae, they are allowed to hatch under optimal conditions (30-35° C., about 50-90% humidity, ideally 60-80% humidity). Adult flies are allowed to mate in green houses (at a temperature of between 25° and 35°, ideally 27° C., at 60-90% humidity) and eggs collected, and the eggs hatched, and then grown on a defined, prepared medium until they become visible before inoculation into the digester. As discussed above, larvae become visible (when in their cultivation medium) when they are at least 0.5 mm in length, such as about 1 mm in length, which length they typically achieve about 5 days after hatching, although larvae may reach a suitable length for inoculation earlier or later than 5 days depending on the conditions under which they are grown (nutrition, temperature, humidity), and may be ready for inoculation in as few as 3 days, or may be ready as late as, for example, ten or even eleven days after hatching.

### Processing of Mixed Solids Waste

A process according to the present invention for treating MSW utilises a digester, comprised of an extended tubular structure which may be circular, oval, oblong or square in cross-section. In certain embodiments the digester is substantially cylindrical. As is the case with large-scale treatments such as the present invention relates to, the digester may be several metres in length, even 20 m or more in length, and have a cross-sectional area of the order of from about 2 m² to about 50 m² or more.

The digester may comprise internal baffles, which may be configured in any way as known in the art, such as axially extending, radially extending, helical, etc. A helical baffle arrangement may assist in moving the MSW from the entrance towards the exit of the digester, such that the mean residence time for any given MSW in the digester is from about 1 day to about 10 days, such as from about 2 days to about 8 days, from about 2 days to about 5 days, from about 2 days to about 4 days, about 2 days, about 3 days, about 4 days, or about 5 days.

An alternative, or complementary means to encourage movement of the MSW down the digester as it is treated, is to configure the digester to be disposed at a descending angle from the entrance to the exit of the digester. The angle of disposition may be from about 1° to about 10°, such as from 1° to about 8°, from about 1° to about 6°, from about 2° to about 5°, from about 2° to about 4°, about 1°, about 2°, about 3°, about 4°, about 5°, about 6°, about 7°, or about 8°.

To maintain sufficient aeration (even remove the need for any forced aeration), assist in drying of the MSW during treatment, increase access of dipteran larvae to biological organic waste, and to assist in encouraging the MSW to move down the digester from the entrance to the exit, the digester may be axially rotated. Too high a rotational speed may be deleterious to the dipteran larvae, but such speed (for example, 6 rpm) is unlikely to be relevant to processes according to the present invention. According to certain embodiments the digester is rotated axially at a speed of from about 1 revolution per day to about 50 revolutions per day, about 1 revolution per day to about 40 revolutions per day, about 2 revolutions per day to about 40 revolutions per day, about 2 revolutions per day to about 35 revolutions per day, about 2 revolutions per day to about 30 revolutions per day, about 5 revolutions per day to about 30 revolutions per day, about 10 revolutions per day to about 30 revolutions per day, about 15 revolutions per day, about 20 revolutions per day, about 25 revolutions per day, or about 30 revolutions per day.

MSW may be introduced into the digester at rates determined based on at least the biodegradable organic matter content of the MSW, the planned mean residence time of the MSW in the digester (which may also be in part governed by any axial rotation speed of the digester), and the BSF inoculation rate. In addition, if the operational fill rate is too low, the process becomes inefficient, whereas if the digester is too full at any time, the treated mixture becomes anaerobic and too moist, although air could be forced into the digester via blowers, but this introduces extra costs. Broadly speaking the digester should operate at about 20% to about 70% capacity, such as from about 25% to about 60% capacity, from about 30% to about 50%, from about 30% to about 45% or from about 30% to about 40% capacity. Fill rates can be increased, or batches of MSW undergoing treatment might be rescued, if additional air is forced into the digester via, for example, blowers.

Depending on the mean residence time of MSW in the digester (as determined by rotational speed and/or angle of disposition of the digester), operational levels of MSW in the digester may be achieved by introducing MSW into the entrance of the digester at a rate of from about 0.1 to about 0.5 volumes (i.e. digester volumes) per day, such as from about 0.1 to about 0.45 volumes per day, from about 0.1 to about 0.4 volumes per day, from about 0.1 to about 0.3 volumes per day, from about 0.1 to about 0.25 volumes per day, from about 0.1 to about 0.2 volumes per day, about 0.1 volumes per day, about 0.15 volumes per day, about 0.2 volumes per day, about 0.25 volumes per day, about 0.3 volumes per day, about 0.35 volumes per day, or about 0.4 volumes per day.

Rate of inoculation of BSF larvae into the digester will also be determined based on the digestible organic matter content of MSW fed into the digester, the rate of MSW feed into the digester, and the planned mean residence time of the MSW in the digester. It is very difficult to estimate the actual mass of BSF larvae actually being used to inoculate the digester, as when they first become properly visible they are about 1 mm in length, and are not readily separable from the medium they are in. It is more facile, therefore, to relate the inoculation rate based on the amount of eggs that are used to raise the BSF larvae for inoculation (and, if the amount that hatch are more or less than required, this is not serious, as either the digestible organic material will be more fully digested, or the stream exiting the digester will still comprise digestible organic matter, which can still be used as a refuse-derived reductant). Thus, in certain embodiments BSF larvae are inoculated into the digester based on egg weight resultant larvae per tonne (“EGRL/T”) digestible organic matter, that is, the weight of BSF eggs used to hatch larvae for the digester per tonne of digestible organic matter in the MSW to be treated. Broadly speaking, BSF larvae may be inoculated into the digester at a rate of from about 2.5 g EGRL/T to about 100 g EGRL/T digestible organic matter, such as about 5 g EGRL/T to about 75 g EGRL/T, about 5 g EGRL/T to about 50 g EGRL/T, about 5 g EGRL/T to about 40 g EGRL/T, about 5 g EGRL/T to about 30 g EGRL/T, about 5 g EGRL/T to about 20 g EGRL/T, about 10 g EGRL/T to about 20 g EGRL/T, about 5 g EGRL/T, about 10 g EGRL/T, about 12 g EGRL/T, about 15 g EGRL/T, about 20 g EGRL/T, or about 25 g EGRL/T digestible organic matter.

With most MSW batches tested to date, there appears to be no need to supplement the MSW with any micro- or macronutrients, or moisture, in fact part of the residence time in the digester is often to get moisture levels down. However, micro- and/or macro nutrients, as well as moisture may be added to MSW under treatment in the digester if necessary.

Temperature and humidity are significant concerns for most insect/dipteran larvae. Although humidity is unlikely to be a problem when dealing with most MSW sources, if necessary, moisture can be added to the digester through sprayers, or the material can be dried out more by extending its residence time in the digester and/or increasing air flow through the digester (through use of blowers, or a lower MSW feed rate, for example). Temperature, on the other hand, can be a significant issue in digesters carrying out processes according to the present invention. BSF larvae, for example, require a temperature between about 15° C. and 60° C. for survival, and do not eat outside a temperature range of about 15 to about 46° C., so heating (using elements surrounding the digester) or cooling (via blowers/fans) may be required depending on the location. Ideally the temperature in the digester will be between 20° C. and 40° C., such as between 25° C. and 40° C., between 25° C. and 35° C., about 25° C., about 30° C. or about 35° C.

Product Streams from Digested MSW

The biodegradation digester processes according to the present invention may result in at least three product streams: matured or at least advanced dipteran larvae; composted organic material; and non-digested material, which will be mostly comprised of small and/or soft plastic materials, rubber, wood, etc.

As mentioned earlier, BSF larvae will self-harvest when they have matured and are ready to pupate by migrating to the top, and out of the substrate, so most of them will migrate to the entrance of the digester, where a collector funnels the BSF larvae to a collection bin for processing. Some BSF larvae go through the digester and come out the exit. At the exit, the treated MSW is passed through a screen, such as a rotary screen with a 25 mm mesh, which substantially all composted organic material and larvae will pass through, but larger non-digested materials such as plastics, rubber and wood pieces, will not, and that stream is redirected for further processing, for example to produce refuse derived reductant (RDR; discussed later). The composted organic material and larvae are then passed through a suitable screen, such as a 10 mm or 5 mm screen (such as a vibrating screen), which most larvae will not pass through, thereby separating the larvae and the composted organic streams.

While a certain amount of the BSF larvae may be used to lay eggs for the next batch(es) of BSF larvae for inoculation of digesters, the majority of the BSF larvae may be used as food/feed directly, or be processed to prepare food/feed (such as pelleted stock feed). If not being used or processed immediately, the BSF larvae may be washed, and then killed (for example by steam) and stored appropriately (e.g. frozen).

BSF larvae that are ready to pupate are, generally speaking, of slightly lower nutritive value than more immature larvae—if a higher nutritive quality BSF larva product desired, larvae may be harvested from MSW under treatment before they migrate by themselves out of the digester. This can be achieved by increasing throughput (volume per day) by rotation and increasing BSF larvae inoculation amounts. While the biodegradable organic matter in the MSW will be fully digested, this will result in a larger proportion of adolescent larvae being harvested early and forced through to the discharge end of the digester. The negative side of this is that it will reduce the amount of mature BSF larvae self-harvesting, but if the higher protein larvae can be sold at a premium, this may be feasible.

The composted organics stream may be used to manufacture fertilisers or potting mixes or be used directly as compost. Alternatively, the composted organics stream may be directly drilled back into soil, such as agricultural soil, as a soil improver/conditioner.

Overall, almost all product streams resulting from the digester are utilised, resulting in minimal residual matter to be disposed of.

Preferred forms of the present invention will now be described, by way of example only, with reference to the following examples, including comparative data, and which are not to be taken to be limiting to the scope or spirit of the invention in any way.

### Example 1

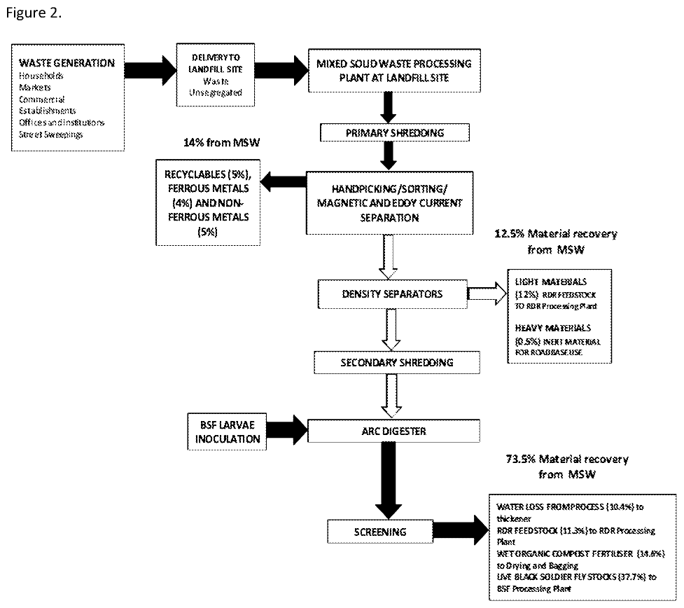

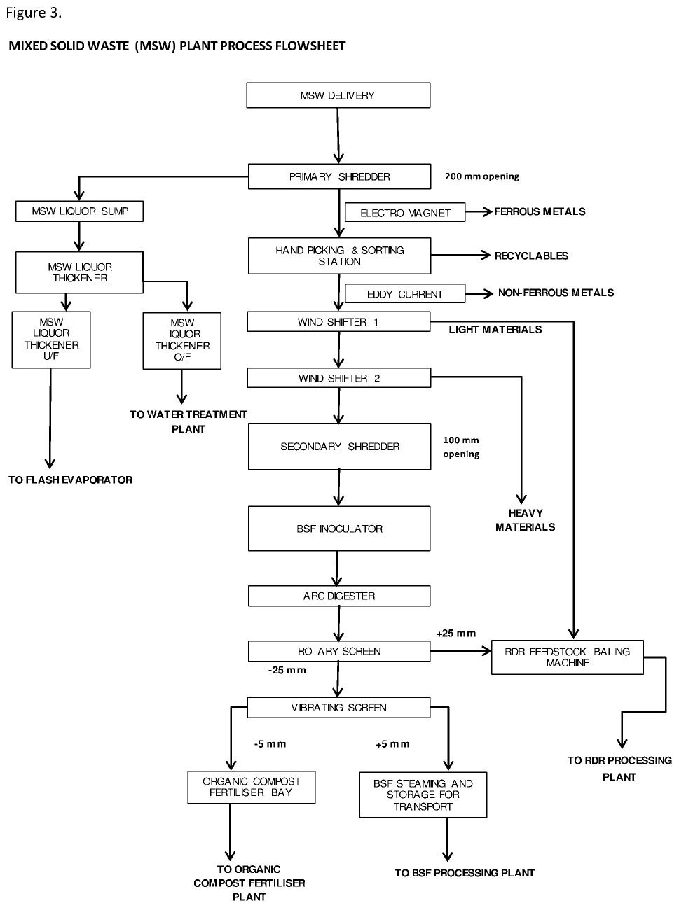

Having reference to FIGS. 2 to 4, a preferred embodiment of the processes of the present invention is as follows.

A digester comprised of a metal cylinder (in the treatment facility this is about 16 m in length, with a cross-sectional area of about 20 m² is disposed at a descending angle of between 2° and 3° from entrance to exit. The digester is axially rotated at a rate of between 6 and 24 revolutions per day and shredded MSW (biodegradable organic matter content of about 50%) which has had metals, glass and recyclable plastic materials removed, is continuously fed into the digester via a conveyor belt (from a secondary shredder) at a rate sufficient to keep it about 40% full and to maintain a mean residence time of the MSW in the digester of about 3 days. 5-day old BSF larvae (raised in an annexed hatching facility) are inoculated into the digester a rate of about 1 g BSF eggs originally used to hatch the larvae per 5 kg of MSW to about 1 g BSF eggs per 50 Kg MSW.

As BSF larvae mature, they migrate to the surface and out of the digester into collection means below the upper lip of the digester, and the BSF larvae are then directed to a collection bin.

Treated MSW leaving the digester is conveyed to a rotary 25 mm screen where the stream is separated into two streams—one comprising mostly larger plastic, rubber and wood materials, which can be further processed into either RDF or RDR, and the ‘under 25 mm’ stream comprising mostly composted organic matter and BSF larvae. The latter stream is then conveyed to a vibrating 10 mm or even 5 mm screen, where it is separated into two more streams, one (>10 mm or 5 mm fraction) comprising mostly BSF larvae, which is combined with the self-harvested BSF larvae, and the other (<10 mm or 5 mm fraction) comprising composted organic matter, which can then be used for composting soils, as fertiliser, as a component of potting mixes, or can be drilled directly into soil as a soil improver/conditioner.

### Example 2

In a specific working example, a cylindrical digester 16 m long with a diameter of 5 m (total volume 314.2 m³), was disposed at a descending angle of 3° and rotated at a speed of 24 revolutions per day.

Mixed solids waste (of a bulk density of about 410 kg/m³) which had been processed by two shredders, and large recyclable materials (such as large metallic and plastic materials) removed, and with a biodegradable organic matter content of about 60% and digestible organic matter of about 27% was fed into the entrance to the digester at a rate of about 892 kg per hour (about 232 kg digestible organic matter per hour; about 52.4 m³/day, or about 0.17 volumes per day), based on a mean residence time of the MSW in the digester of 3 days, with the digester being run at about 50% volume fill (therefore operating volume of about 157.1 m³).

The digester was inoculated with larvae resulting from about 20 g BSF eggs per hour (ie. 20 g EGRL per hour), and therefore about 45 g EGRL/T MSW (or 12 g EGRL/T digestible organic matter).

Mature or maturing BSF larvae were self-harvested by allowing the BSF larvae to migrate out of the entrance of the digester into a collection bin, and also harvested from the treated MSW exiting the lower end of the digester. Treated MSW collected at the exit of the digester was first screened via a 25 mm rotary screen (to remove plastics and other large solids which were routed to RDR production—see below), and the screened material then passed through a 5 mm vibrating screen to remove BSF larvae, with the screened material being predominantly biodegraded organics.

As a result of this treatment of the MSW, about 75% of the overall moisture content was removed, 38.7 kg BSF larvae were harvested per hour, and about 313 kg biodegraded organics (which may be used as mulch, compost or fertiliser) were harvested per hour.

### RDR Process

A major by-product of the process described above is the non-compostable plastic portion of the original MSW.

Plastic is simply a solid polymerised hydrocarbon consisting primarily of carbon and hydrogen. A target of the process according to the invention is to recover the carbon and hydrogen that make up waste plastic, as this has varied industrial applications and commercial value. An advantage of using plastics for this purpose is that, as the material has been processed from its original crude oil source, it has typically been cleaned of pollutants such as sulphur and nitrogen, which are normally present in coal and coke.

Similarly, any organic material present in the plastic waste is essentially made up of carbon and hydrogen. This makes them compatible with the product formed from plastic as they are applied in the same industrial processes.

The present invention seeks to utilise the plastic waste, substantially separated form organic waste via the above described process, into Refuse Derived Reductant (RDR) suitable for use as a replacement for metallurgical coke and high grade coal used for steel making and other applications requiring high fixed carbon content and calorific value.

RDR is a different concept to an existing product called RDF (Refuse Derived Fuels). The latter requires certain properties and parameters enhanced, hence the extra processing requirement, to optimize the carbon content of the product. It also addresses the limitations in industries where the product can be used. RDFs are simply a raw product scavenged from the mixed solid waste stream which is primarily used as a mixed fuel with no means to control quality and consistency.

The process of producing RDR enables the product to be managed for consistency and quality which is a key element in its marketability. As it goes through a manufacturing stage there is opportunity control the finished product so that they meet specifications.

All the oversize material screened after the ARC digester is transferred to an RDR conversion step where the material first passes through a drying and semi-pyrolysing process to remove excess moisture and start charcolising the organics. The temperatures in the chambers are managed to ensure the plastics soften but do not liquefy. This is important as if the plastics start to vaporize then the carbon and hydrogen will be lost as a gas if not utilized.

The softened plastic is then put through a plastic extruder to form it into RDR briquette, dried and stored for shipment.

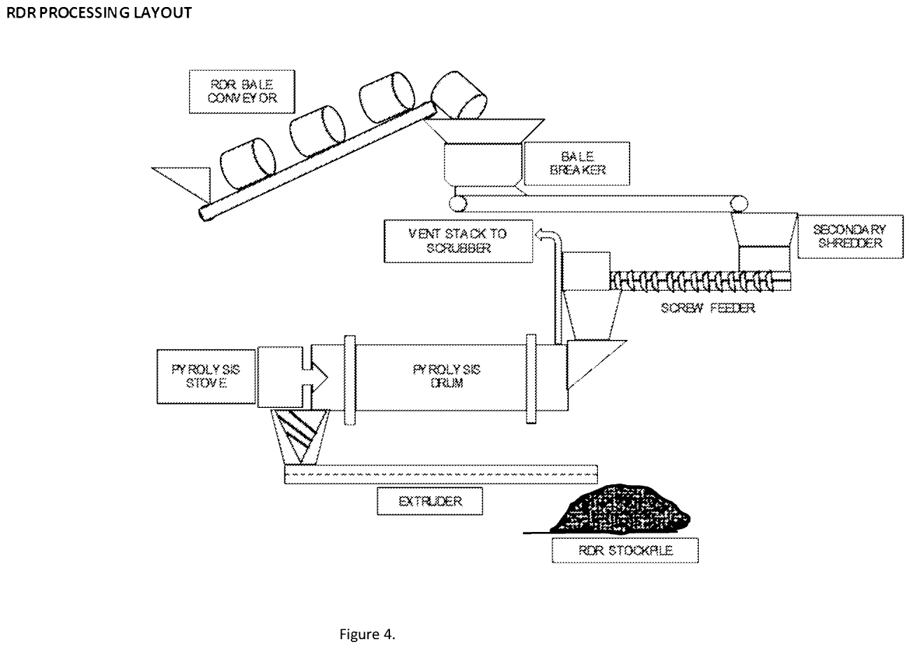

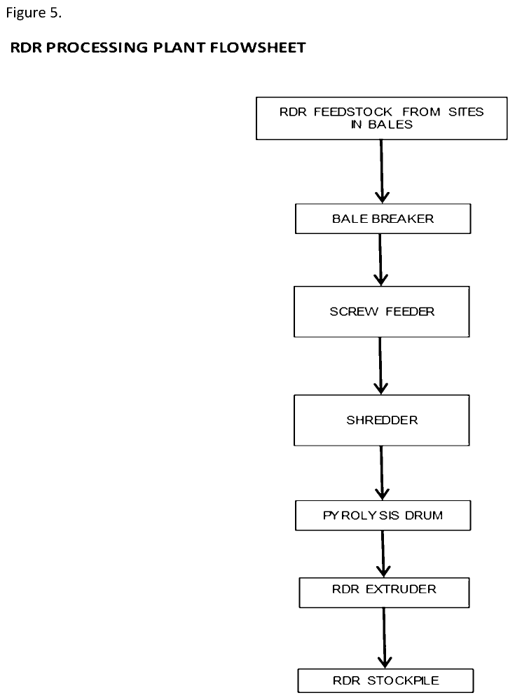

Turning to FIGS. 4 and 5, there are disclosed simplified flowcharts for an RDR process according to the invention.

As will be seen in the diagram, all the oversize material screened after the ARC digester is transferred to an RDR conversion step. Before proceeding to the RDR conversion step, the oversize material is processed first in a baling machine to produce RDR feedstock bales.

In the RDR processing plant, the RDR feedstock bales are transported to the bale breaker using a conveyor belt. Alternatively, the RDR feedstock bales may also be transported to the bale breaker via other techniques like mechanical or manual dumping, as required.

In the bale breaker, the RDR bales are opened and transferred to the 200 mm shredder by conveyor. The size of the RDR feed stock is then reduced to approximately 25 mm through a secondary shredder to match the top size of RDR feedstock harvested from the BSF rotary screen, wherein +25 mm is RDR feedstock and −25 mm is BSF organics.

The shredded RDR feedstock is transported to the hopper of a pyrolysis drum using a screw feeder, where the RDR feedstock is dried and semi-pyrolysed to remove excess moisture. The dried RDR feedstock is subjected to a process of pyrolysis at a temperature between 100° C. and 200° C., in the substantial absence of oxygen to avoid formation of carbon monoxide or the oxidation of the plastic material. The temperature in the pyroliser should not exceed 200° C.

The temperature in the pyrolysis drum is managed to ensure the plastics/composite materials soften but do not liquefy. This is important as if the plastics start to vaporize then the carbon and hydrogen will be lost as a gas if not utilized.

The plastics/composite material is then transferred from the pyrolysis drum to an extruder (which may be single or twin screw). The transfer of the material to the extruder feed is sealed in order to maintain the pyrolysis temperature and pressure—it is important not to allow the plastic to vent to atmosphere at this stage.

The softened plastic passes through the extruder to form it into briquettes, which are cut as they emerge from the extruder and dried and stored for shipment.

The temperature in the extruder barrel is maintained at between 100° C. and 200° C. and the pressure remains high due to the mechanical action of the screw(s).

By condensing the RDR feedstock into extruded briquettes the material density is increased and facilitates handing and shipment. It also makes it more applicable in varied industrial applications.

As mentioned above, RDR (Refuse Derived Reductant) is a vastly different concept to an existing product called RDF (Refuse Derived Fuels). The latter requires certain properties and parameters enhanced, hence the extra processing requirement, to optimize the carbon content of the product. It also addresses the limitations in industries where the product can be used.

RDFs are simply a raw product scavenged from the mixed solid waste stream which is primarily used as a mixed fuel with no means to control quality and consistency.

The process of producing RDR enables the product to be managed for consistency and quality which is a key element in its marketability. As it goes through a manufacturing stage there is opportunity control the finished product so that they meet specifications.

### Example 3

Turning to FIG. 4, there is illustrated an RDR process as per the invention.

Bales of RDR feedstock material from the BSF process, composed of about 45% plastic material, the remainder being textiles, wood, rubber/leather and remaining organics, are fed into a bale-breaker where they are reduced to about 200 mm size. This is then fed into a secondary shredder which reduced the particle size to about 25 mm.

A closed screw feeder conveys the shredded feedstock to the rotary pyrolysis drum feed hopper. The material fed into the rotary pyrolysis drum is dried and partially charcolised as it passes through. The internal pyrolysis drum temperature is maintained at an average of 200° C. with a low-oxygen atmosphere to allow the organic material content to start charcolising and igniting whilst the plastic content begins to liquefy.

The partially charcolised and liquefied material is then discharged directly into a single-screw RDR extruder via a vertical auger screw. The pyrolysis drum exit and the extruder feed are enclosed and the low oxygen conditions are maintained throughout the transfer.

The material is then caught by the extruder screw and made malleable with the high temperature and pressure generated inside the extruder screw of around 200° C. and 5000 psi. The composite plasticised material consisting of plastics, rubber, wood, inorganics, textiles, paper, cardboard and leather are then extruded into RDR tubes. The tubes are broken into shorter stems then cooled and stockpiled.

The RDR tubes have successfully been used as a reductant carbon source in the manufacture of calcium carbide.

### Process Integration

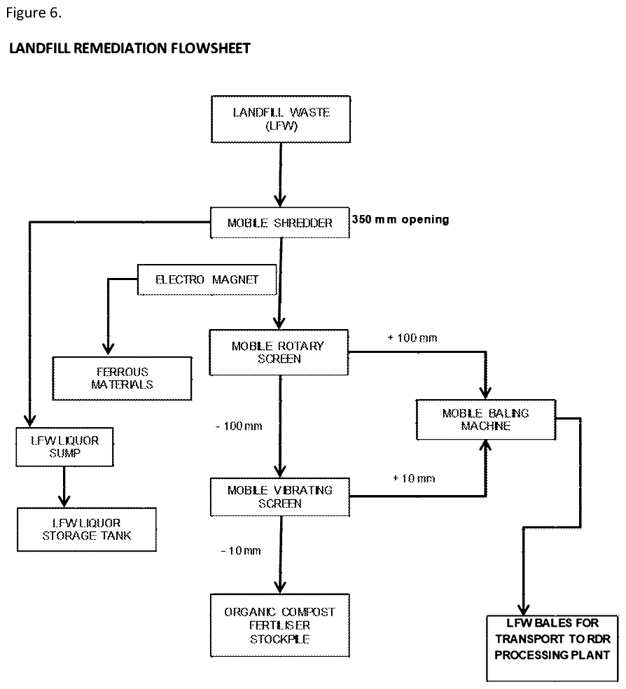

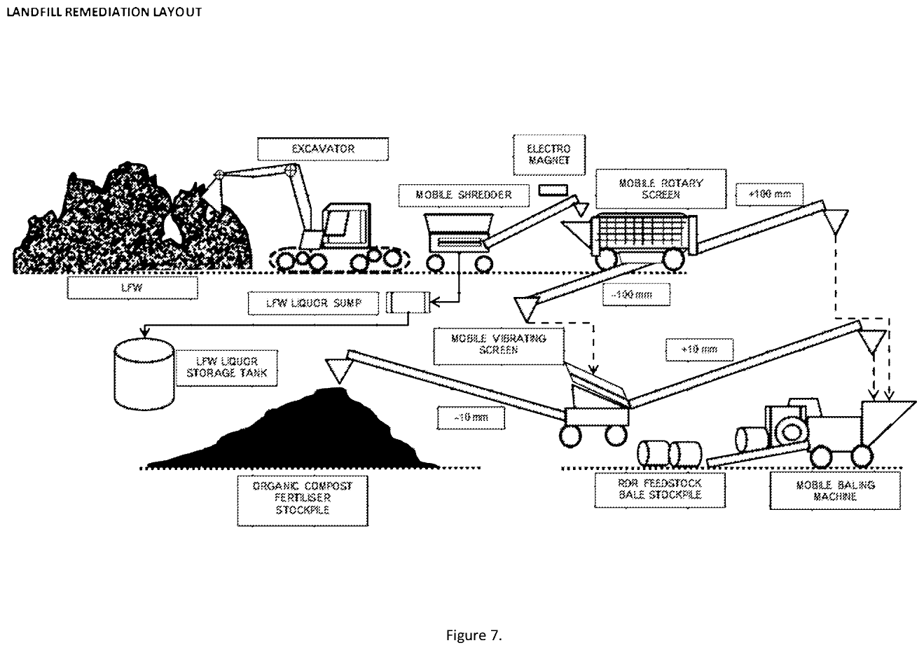

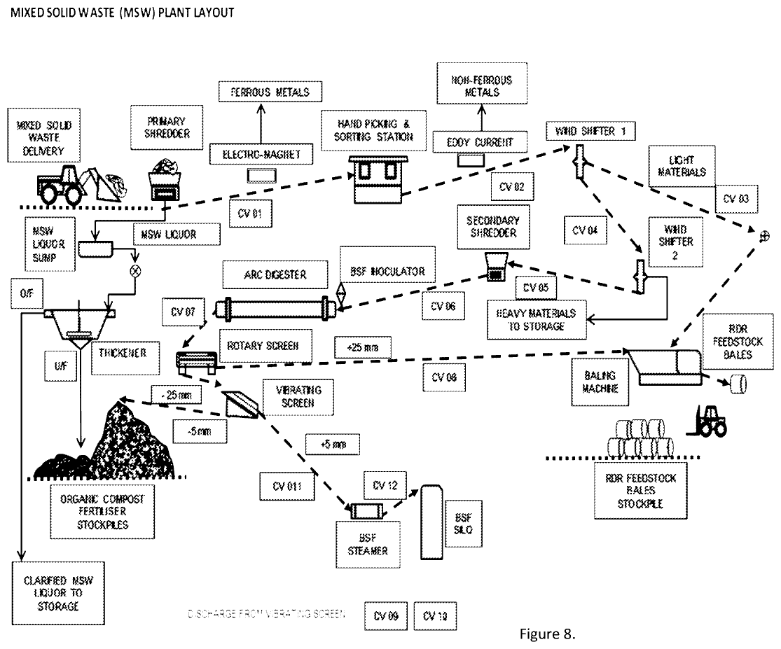

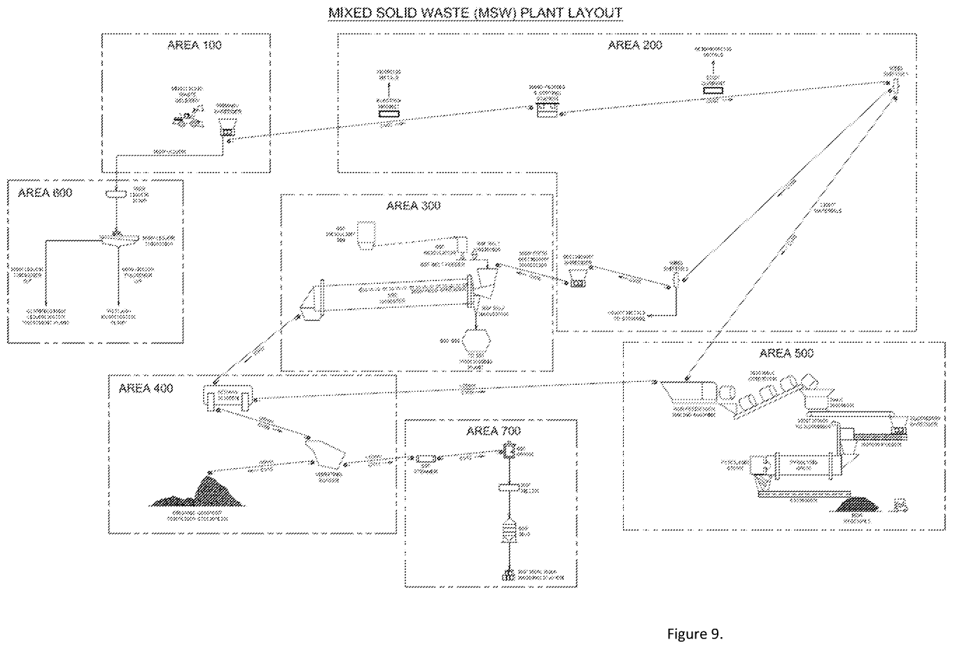

Turning to FIGS. 6, 7, 8 and 9, there are shown a process flow and layout diagrams for a landfill remediation operation according to the present invention, where both organic material processing and RDR manufacture from the resulting plastic material are integrated in a single continuous operation to extract useful products from MSW on site.

This is a major development in the efficient continuous processing of MSW into useful products in a practical manner.

It will be appreciated by those skilled in the art that the above described embodiment is merely one example of how the inventive concept can be implemented. It will be understood that other embodiments may be conceived that, while differing in their detail, nevertheless fall within the same inventive concept and represent the same invention.

1. A method for treatment of mixed solids waste, wherein the waste comprises 10% to 80% organic matter, comprising:
continuously introducing the mixed solids waste and a plurality of dipteran larvae into an entrance of a digester vessel;
continuously collecting treated reduced organics waste at the exit of the digester vessel; and
continuously harvesting dipteran larvae at one or more desired locations along the digester vessel.

2. The method of claim 1, wherein the plurality of dipteran larvae are a species of Stratiomyidae family of flies.

3. The method of claim 2, wherein the plurality of dipteran larvae are of black soldier flies.

4. The method of claim 1, wherein the plurality of dipteran larvae are five days old at inoculation.

5. The method of claim 1, further comprising:
continuously shredding the mixed solids waste at least once; and
continuously separating recyclable metallic, glass, and plastic materials from the mixed solids waste.

6. The method of claim 1, wherein the digester vessel is a cylindrical vessel disposed at a descending angle between 1° and 6°, and is axially rotated at a speed of between 1 and 50 revolutions per day.

7. The method of claim 1, wherein the digester vessel is maintained at a fill level of between 15% and 60%.

8. The method of claim 16, wherein the mixed solids waste comprises 40% to 60% organic matter.

9. The method of claim 16, wherein the mixed solids waste comprises 15% to 50% digestible organic matter.

10. The method of claim 1, wherein the mixed solids waste has a mean residence time of approximately 2 days to 5 days in the digester vessel.

11. The method of claim 1, wherein the mixed solids waste is introduced into the entrance of the digester vessel at a rate of between 0.05 and 0.3 volumes per day.

12. The method of claim 1, wherein a rate of inoculation of the plurality of dipteran larvae is adjusted for a level of digestible organic matter in the mixed solids waste and residence time of the mixed solids waste in the digester vessel.

13. The process of claim 1, wherein the plurality of dipteran larvae are introduced at the entrance of the digester vessel at a rate of between 5 and 25 egg weight resultant larvae per ton of the digestible organic matter.

14. (canceled)

15. (canceled)

16. (canceled)

17. (canceled)

18. (canceled)

19. A method of producing a metallurgical reductant material from municipal waste, wherein the municipal waste comprises at least 10% plastic material, comprising:
shredding the municipal waste;
pyrolysing the municipal waste at a pyrolysis temperature of between 100° C. and 200° C.;
extruding the municipal waste in an extruder at an extrusion pressure and an extrusion temperature and in absence of oxygen; and
reducing the municipal waste into pellets.

20. The method of claim 14, further comprising the municipal waste drying.

21. The method of claim 14, wherein the pyrolysis temperature is 200° C.

22. The method of claim 14, wherein extrusion temperature is between 100° C. and 200° C.

23. The method of claim 14, wherein the extrusion pressure is between 3,000 psi and 10,000 psi.

24. (canceled)

25. The method of claim 14, further comprising separating the plastic material from the municipal waste.

26. (canceled)

27. (canceled)

28. A method of processing municipal waste comprised of plastic matter and organic matter, comprising
continuously digesting the organic matter in the municipal waste into a useful protein meal; continuously processing the plastic matter and remaining organic material into refuse derived reductant.

29. (canceled)

30. (canceled)
